(19) United States
(12) Patent Application Publication
(10) Pub. No.: US 2021/0252216 A1
(43) Pub. Date: Aug. 19, 2021

(54) SMARTPHONE-CONTROLLED IMPLANTABLE NEURAL DEVICES FOR LONG-TERM WIRELESS DRUG DELIVERY AND LIGHT STIMULATION, AND OPERATING METHOD THEREOF

(71) Applicants: Korea Advanced Institute of Science and Technology, Daejeon (KR); Washington University, St. Louis, MO (US)

(72) Inventors: Jae-Woong Jeong, Daejeon (KR); Michael R. Bruchas, St. Louis, MO (US); Raza Qazi, Daejeon (KR); Adrian M. Gomez, St. Louis, MO (US)

(73) Assignees: Korea Advanced Institute of Science and Technology, Daejeon (KR); Washington University, St. Louis, MO (US)

(21) Appl. No.: 16/983,910

(22) Filed: Aug. 3, 2020

(30) Foreign Application Priority Data
Feb. 14, 2020 (KR) 10-2020-0018105

(51) Int. Cl. A61M 5/142; A61N 1/372; A61N 1/378; A61N 5/06; A61M 5/168; G08C 17/00; G08C 23/04

(52) U.S. Cl. CPC A61M 5/14276; A61N 1/0529; A61N 1/37247; A61N 1/378; A61N 5/0601; A61N 1/37252; A61M 5/16804; G08C 17/00; G08C 23/04; A61N 2005/0612; A61N 2005/0626; A61M 2205/04; A61M 2250/00; A61M 2210/0693; A61M 2205/8206; A61M 2205/3584; A61M 2205/52; A61M 2205/051; A61M 2205/054; A61N 1/37217

(57) ABSTRACT

Various embodiments, which relate to an electronic device implanted in tissue of an animal and for delivering stimulation to the neural tissue and operating method thereof, may be configured to generate a control instruction based on a control signal wirelessly received from an external device, and based on the control instruction, to input stimulation through a neural probe formed with flexible material and led out to a predetermined location of the tissue. According to various embodiments, the stimulation includes chemical stimulation by a fluid type of drug, and the neural probe may be formed to flow drug, and may include at least one fluid tube for inputting the chemical stimulation by being opened at one end of the neural probe.

### CROSS-REFERENCE(S) TO RELATED APPLICATION(S)

This application claims the priority benefit of Korean Patent Application No. 10-2020-0018105, filed on Feb. 14, 2020, Korean Intellectual Property Office, the disclosure of which is incorporated herein by reference.

### BACKGROUND

### 1. Field of the Invention

The following example embodiments relate to an electronic device and operating method thereof, more particularly, a stand-alone, smartphone-controlled, implantable neural device capable of long-term wireless drug delivery and light stimulation, and operating method thereof.

### 2. Description of Related Art

Optogenetics and pharmacology are novel methods that can precisely control target neurons or any cell type without affecting surrounding cells using light or drug or a combination of the light and drug. Since these may precisely and selectively control the target neural circuits with higher spatiotemporal resolution than traditional electrical stimulation methods, they recently gained popularity as among the most accurate and reliable tools for brain research and treatment of neurodegenerative diseases.

Conventional optical fibers and metal cannulas used respectively for light and drug delivery are relatively bulky to implement within a single multifunctional probe control, making it hard to control same neural circuits selectively with minimal invasion, thus exacerbating tissue damage and inflammation. Also, since the existing devices are manufactured using stiff materials such as silica, metal, and the like, it creates a large mechanical characteristic mismatch between the soft tissue and a stiff implanted device, further aggravating inflammatory response and creating neuroglial scarring or aganglionosis phenomenon, thus making it unsuitable as a long-term implant. Moreover, since the existing tethered devices should be connected to large and bulky external equipment with multiple wirings and tubes, after being implanted in soft and sensitive tissue of animals, it greatly restricts free behavior and movement of animals, thus preventing reliable artifact-free studies of complex behavior or neural functions within natural environments.

Recently, tether-free standalone implantable devices using infrared and radiofrequency have been developed in order to overcome above limitations. However, the infrared devices have significant limitations in range and line of sight with limited wireless features, therefore difficult to control them reliably in complex studies and setups. The radiofrequency devices, though offering some benefits over infrared, are also not reliable enough especially in studies involving movement of animals due to limited working range and susceptibility to radiofrequency signal orientation and polarization.

### SUMMARY

Embodiments of the inventive concept provide an implantable electronic device capable of elaborately delivering multimodal stimulation to a specific location of tissue of an animal and operating method thereof.

Embodiments of the inventive concept provide an electronic device implanted in a ‘single surgical step’, which is capable of minimizing tissue damage and inflammatory response of an animal, both during surgical process and also after being implanted in tissue of the animal for long periods of time and operating method thereof. Embodiments of the inventive concept provide an electronic device capable of being used long-term while implanted in tissue of an animal and operating method thereof.

Embodiments of the inventive concept provide an electronic device implanted in tissue of an animal and for delivering multiple modes of stimulation to the neural tissue and operating method thereof.

According to an exemplary embodiment, an electronic device may include a probe module including a neural probe formed with flexible material and led out to a predetermined location in the tissue, and configured to input specific stimulation to the location through the neural probe, and a wireless control module configured to be connected to the probe module and generate control instructions to customize one or more parameters for each mode of the stimulation.

According to an exemplary embodiment, the stimulation may include chemical stimulation by a fluid form of drug, and the neural probe may include at least one fluid tube for inputting the chemical stimulation to the location by precisely controlled drug flow which then comes out from one end of the neural probe which lies next to the target tissue location.

According to an exemplary embodiment, the stimulation may include optical stimulation by light, and the probe module may further include at least one light-emitting element for generating the optical stimulation on the location based on the control instruction, by being mounted on one end of the neural probe which lies next to the target tissue location.

According to an exemplary embodiment, an operating method of an electronic device may include processing and/or decoding a control instruction based on a user signal wirelessly received from an external device, such as off-the-shelf smartphone, and generating the specific parameters for one or more stimulation outputs to the target tissue location through an ultra-soft and thin neural probe which is formed with flexible material and led out to the predetermined location of the tissue.

According to an exemplary embodiment, an electronic device is attached to an animal's body, but the ultra-soft and ultra-thin neural probe may be substantially implanted in neural tissue of the animal. As the neural probe is led out to a predetermined location of the neural tissue of the animal, the electronic device may wirelessly receive signals and after processing them, elaborately deliver specific stimulation sequence to the corresponding location. At this time, the neural probe may deliver at least one among chemical stimulation by a fluid form of drug, optical stimulation by light or electrical stimulation by an electrical signal. Selective chemical stimulation may be delivered based on various drugs. Or selective optical stimulation may be delivered based on light in various frequency bands. Or selective electrical stimulation may be delivered based on electrical signals in various frequency bands. In addition, since the neural probe is formed with flexible material and with a width of about 80 μm, even if the neural probe is implanted in the animal's tissue, nervous tissue damage and inflammatory response of the animal may be minimized. Also, a special cartridge module in which the drug is stored is implemented as a ‘plug-n-play’ component which can be detachable and replaceable from the electronic device. Due to this, the electronic device may be used to continuously deliver drugs for a long time while the neural probe stays implanted in the animal's tissue. This ‘plug-n-play’ technique implementation for replaceable drug cartridges resolves the biggest issue in standalone wireless drug delivery—limited drug supply. In addition, since the electronic device may be wirelessly controlled by using an external device such as a smartphone, the use efficiency of the electronic device may be increased. Furthermore, the wireless capabilities of the electronic device may include selective control of a device within a large group of devices in the vicinity, selective output and parameter control within a single device, multi-closed loop control for semi-automation of experiments, long and reliable wireless range (up to 100 m) with log confirmations for all experiments, no line of sight handicap and through-wall device control where a user can control experiments in an adjacent closed room irrespective of his orientation.

These capable wireless features with ultra-compliant and biocompatible probe implant to minimize tissue inflammation, along with its ability to deliver multiple modes of stimulation over long periods of time with minimalistic hardware setup (such as a readily available smartphone) makes it among the most user-friendly and powerful, chronic, tether-free optofluidic devices out there.

### BRIEF DESCRIPTION OF THE DRAWINGS

These and/or other aspects, features, and advantages of the present disclosure will become apparent and more readily appreciated from the following description of embodiments, taken in conjunction with the accompanying drawings of which:

FIG. 1 is an example drawing illustrating a system according to various embodiments;

FIG. 2 is a perspective view illustrating an electronic device of FIG. 1;

FIG. 3 is a block diagram illustrating a neural implant device according to various embodiments;

FIG. 4 is a side view illustrating a neural implant device according to various embodiments;

FIG. 5A is a perspective view illustrating a combined state of a cartridge module and a probe module of FIG. 4;

FIG. 5B is a perspective view illustrating a separated state of a cartridge module and a probe module of FIG. 4;

FIG. 5C is a perspective view illustrating a cartridge module and a probe module of FIG. 4 by disassembling them;

FIG. 5D is an example drawing for illustrating operation features of a cartridge module of FIG. 4;

FIG. 5E is a plan view illustrating a neural probe in area A of FIG. 5A;

FIG. 5F is a plan view illustrating a connected state of a neural probe, a light-emitting element, and a connection terminal in area A of FIG. 5A;

FIG. 6 is a flow chart illustrating an operating method of a neural implant device according to various embodiments;

FIG. 7 is a block diagram illustrating a wireless control device according to various embodiments;

FIG. 8 is a flow chart illustrating an operating method of a wireless control device according to various embodiments; and

FIGS. 9A, 9B, and 9C are example drawings illustrating a user interface of a wireless control device according to various embodiments.

### DETAILED DESCRIPTION

Hereinafter, some example embodiments will be described in detail with reference to the accompanying drawings.

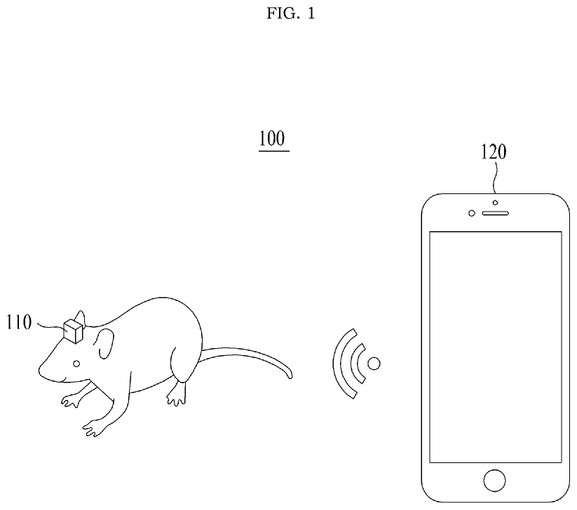

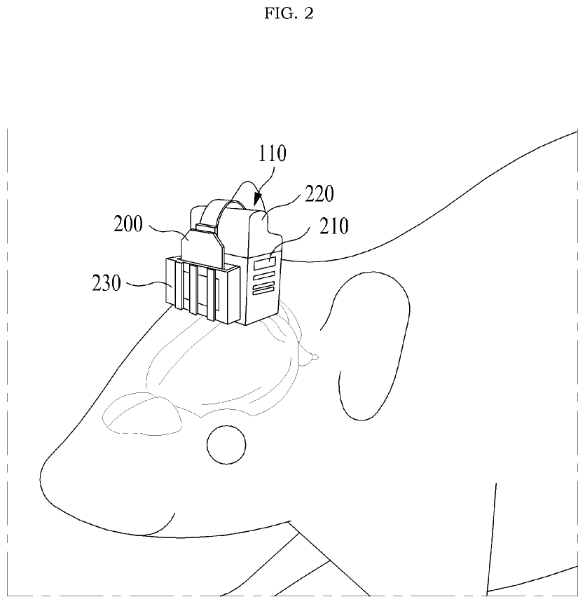

FIG. 1 is an example drawing illustrating a system 100 according to various embodiments. FIG. 2 is a perspective view illustrating an electronic device 110 of FIG. 1.

Referring to FIG. 1, the system 100 according to various embodiments may include various electronic devices 110, 120. The electronic devices 110, 120 may include a first electronic device 110 for delivering stimulation to tissue of an animal and a second electronic device 120 for wirelessly controlling the first electronic device 110. The first electronic device 110 may be driven under control of the second electronic device 120, and deliver stimulation to the tissue of the animal. For example, the stimulation may include at least one among chemical stimulation by a fluid form of drug, or optical stimulation by light or electrical stimulation by an electrical signal. Here, the animal may include human.

The first electronic device 110 may include a neural implant device 200 and housings 210, 220, 230 as shown in FIG. 2. The neural implant device 200 may be implanted in nervous tissue such as brain and the like of the animal. Also, the neural implant device 200 may deliver at least one among chemical stimulation, optical stimulation or electrical stimulation to tissue of the animal. The housings 210, 220, 230 may be fixed on the animal's body and protect the neural implant device 200. For example, the housings 210, 220, 230 may include a first housing 210 directly fixed on the animal's body, a second housing 220 covering the neural implant device 200 by being connected with the first housing 210, and a third housing 230 implemented to be detachable to at least one among the first housing 210 or the second housing 220. Here, even if at least one of components of the neural implant device 200 is exposed from the first housing 210 and the second housing 220, it may be covered by the third housing 230.

The second electronic device 120 may be referred to as a wireless control device 120. For example, the wireless control device 120 may include at least one of a smartphone, a mobile phone, a navigation, a computer, a laptop, a terminal for digital broadcasting, a PDA (Personal Digital Assistants), a PMP (Portable Multimedia Player), a tablet PC, a game console, a wearable device, an IoT (Internet of Things) device, a VR (Virtual Reality) device, and an AR (Augmented Reality) device or a robot.

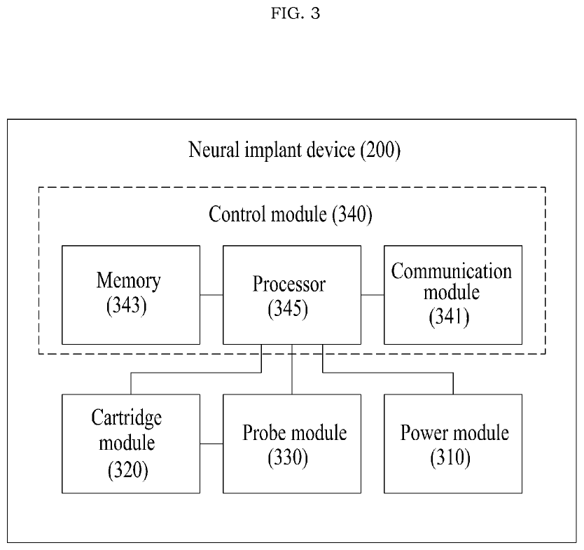

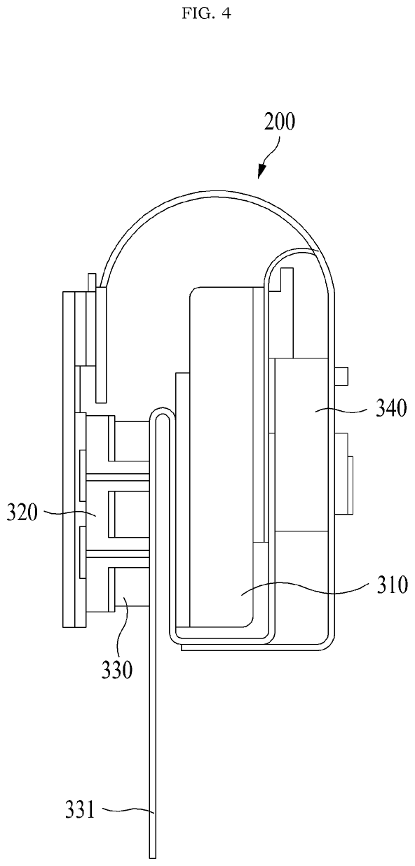

FIG. 3 is a block diagram illustrating the neural implant device 200 according to various embodiments. FIG. 4 is a side view illustrating the neural implant device 200 according to various embodiments.

Referring to FIGS. 3 and 4, the neural implant device 200 of the electronic device 110 according to various embodiments may include at least one among a power module 310, a cartridge module 320, a probe module 330 or a control module 340. In some example embodiments, at least one among components of the neural implant device 200 may be omitted, and at least another one component may be added. In some example embodiments, at least two among the components of the neural implant 200 may be implemented into one integrated circuit.

The power module 310 may manage electric energy supplied to at least one component of the neural implant device 200. At this time, the power module 310 may include at least one among a battery module for storing electric energy, a wireless charge module for wirelessly charging the battery module, or a wireless power management module which enables direct use of wirelessly transmitted radiofrequency energy without the need for the battery. For example, the battery module may include at least one among a primary cell which may not be recharged, a second cell which may be recharged or a fuel cell.

The cartridge module 320 may be storing drug. At this time, the drug may be a fluid form of drug. Also, the cartridge module 320 may be implemented to be replaceable in the neural implant device 200. At this time, the cartridge module 320 may be implemented to be detachable for the probe module 330.

The probe module 330 may be substantially implanted in tissue of an animal, and input stimulation to the tissue of the animal. For this, the probe module 330 may include a neural probe formed with flexible material and led out to a predetermined location of the tissue. The neural probe may input stimulation to the determined location of the tissue through one end. At this time, the one end of the neural probe may contact to the determined location of the tissue. According to one example embodiment, stimulation may include chemical stimulation by drug. The probe module 330 may input chemical stimulation to the determined location of the tissue by using drug supplied form the cartridge module 320. According to another example embodiment, stimulation may include optical stimulation by light. The probe module 330 may input optical chemical to the determined location of the nervous tissue by generating light. According to another example embodiment, stimulation may include electrical stimulation by an electrical signal. The probe module 330 may input the electrical stimulation to the determined location of the nervous tissue by generating an electrical signal. In some example embodiments, the probe module 330 may sense a change for stimulation from the tissue of the animal.

The control module 340 may control at least one component of the neural implant device 200. At this time, the control module 340 may generate a control instruction for generating stimulation. Also, the control module 340 may acquire sensing data from the probe module 330. According to one example embodiment, the control module 340 may include at least one among a communication module 341, a memory 343 or a processor 345.

The communication module 341 may perform communication with an external device, i.e., the wireless control device 120 in the neural implant device 200. The communication module 341 may establish a communication channel between the neural implant device 200 and the wireless control device 120, and through the communication channel, perform communication with the wireless control device 120. At this time, the communication module 341 may perform communication with the wireless control device 120 in near field communication method. For example, the near field communication method may include at least one among BLE (Bluetooth low Energy), Bluetooth, Wi-Fi or IrDA (Infrared Data Association).

The memory 343 may store various data used by at least one component of the neural implant device 200. For example, the memory 343 may include at least one among volatile memory or nonvolatile memory. The data may include at least one program and input data or output data related thereto.

The processor 345 may control at least one component of the neural implant device 200 by executing a program of the memory 343. Through this, the processor 345 may perform data process or operation. The processor 345 may wirelessly communicate with the wireless control device 120 through the communication module 341. For example, the processor 345 may charge the battery module of the power module 310 based on a signal received from the wireless control device 120. The processor 345 may receive a control signal from the wireless control device 120 through the communication module 341. Also, the processor 345 may generate a control instruction based on the control signal. At this time, the processor 345 may generate a control instruction for at least one among the cartridge module 320 or the probe module 330. According to one example embodiment, the processor 345 may transmit the control instruction to the cartridge module 320 to supply drug to the probe module 330. Here, the processor 345 may deliver electric energy to the cartridge module 320. According to another example embodiment, the processor 345 may transmit the control instruction to the probe module 330 in order that the probe module 330 generates light or an electrical signal. Here, the processor 345 may deliver the electric energy to the probe module 330. Also, the processor 345 may acquire sensing data from the probe module 330. Here, the processor 345 may transmit the sensing data to the wireless control device 120. In addition, the processor 345 may store records related to at least one among the control signal, the control instruction or the sensing data, in the memory 343.

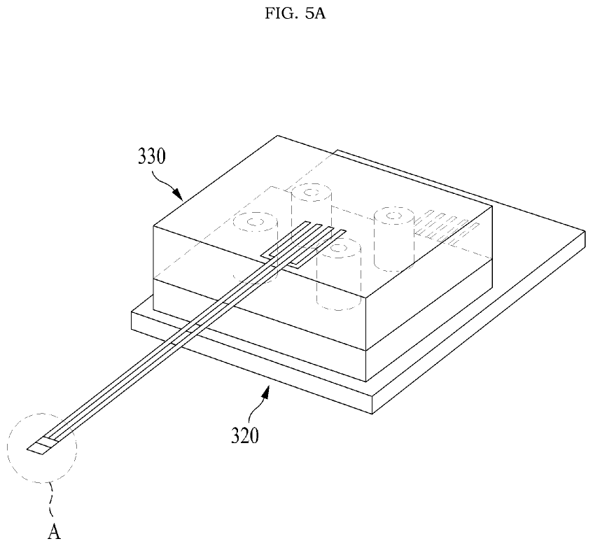

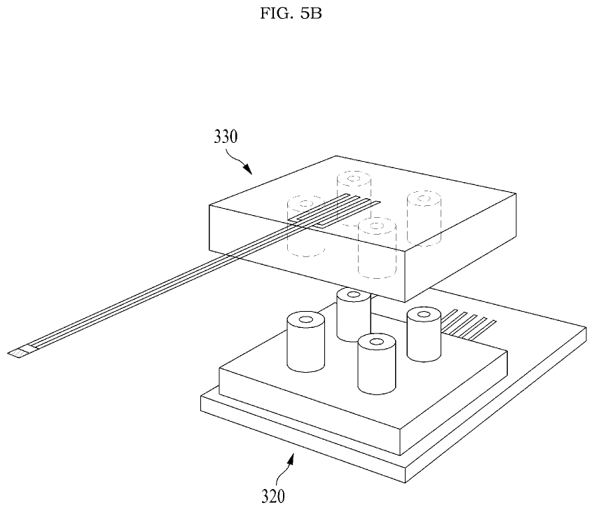

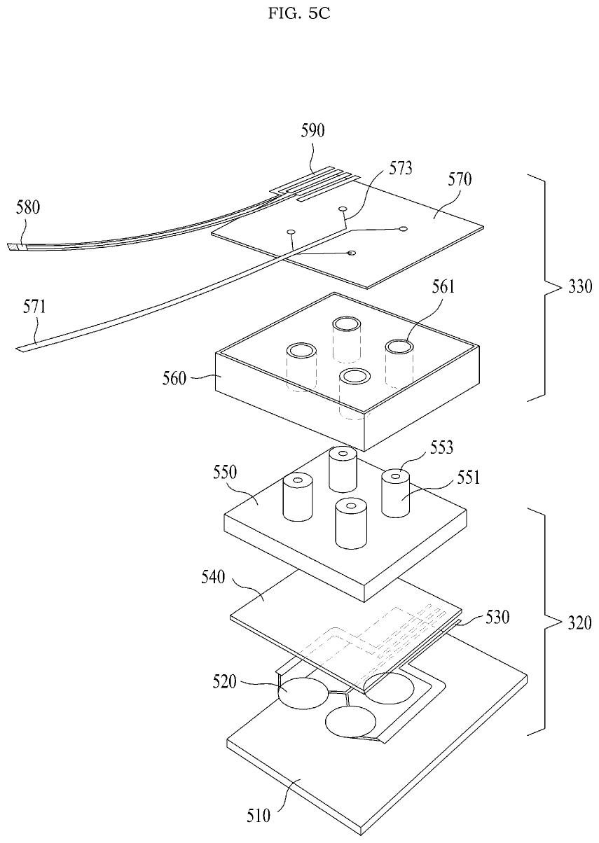

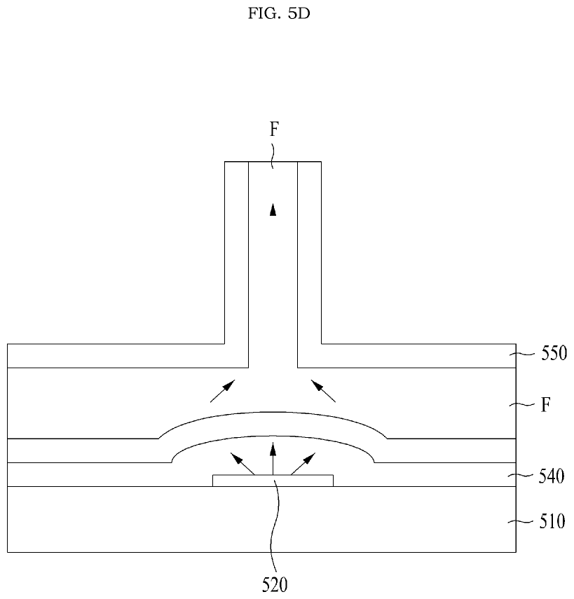

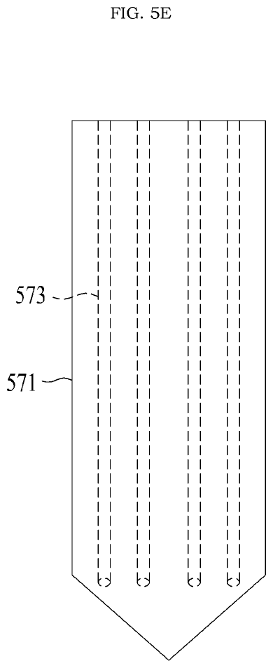

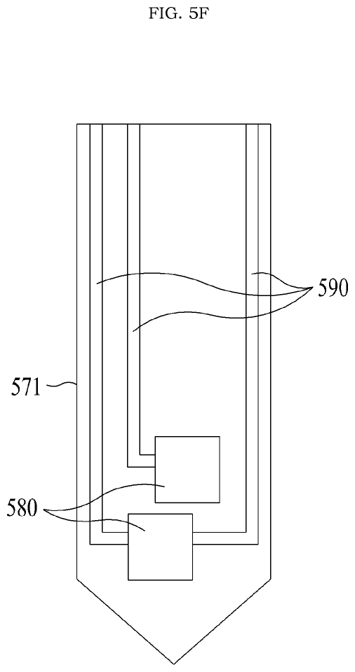

FIG. 5A is a perspective view illustrating a combined state of the cartridge module 320 and the probe module 330 of FIG. 4. FIG. 5B is a perspective view illustrating a separated state of the cartridge module 320 and the probe module 330 of FIG. 4. FIG. 5C is a perspective view illustrating the cartridge module 320 and the probe module 330 of FIG. 4 by disassembling them. FIG. 5D is an example drawing for illustrating operation features of the cartridge module 320 of FIG. 4. FIG. 5E is a plan view illustrating a neural probe 571 in area A of FIG. 5A. FIG. 5F is a plan view illustrating a connected state of the neural probe 571, a light-emitting element 580, and a connection terminal 590 in area A of FIG. 5A.

Referring to FIGS. 5A and 5B, the cartridge module 320 may be detachable to the probe module 330. Through this, during the probe module 330 is implanted in tissue of an animal, the cartridge module 320 may be replaced from the neural implant device 200. For example, the current attached cartridge module 320 is removed from the probe module 330, and a new cartridge module 320 may be attached to the probe module 330. Also, the cartridge module 320 may be implemented with various fluidic pump structures, and supply drug to the probe module 330 based on various fluidic pump methods. For example, the fluidic pump methods may include at least one among a heat-based fluidic pump method, a magnetically-actuated fluidic pump method, a shape memory alloy or shape memory polymer substrate fluidic pump method, or an electrochemistry fluidic pump method.

In some example embodiments, the cartridge module 320 may be implemented to supply drug in the heat-based fluidic pump method as shown in FIG. 5C, and may include at least one among a supporting member 510, at least one heating member 520, at least one connection terminal 530, an expanding member 540 or a drug cartridge 550.

The supporting member 510 may support components of the cartridge module 320.

The heating member 520 may be placed on the supporting member 510. Also, the heating member 520 may generate heat based on a control instruction of the control module 340. At this time, the heating member 520 may be configured with at least one among a predetermined pad or pattern. According to one example embodiment, a plurality of heating members 520 may be arranged on the supporting member 510, and may be spaced apart from each other by a predetermined interval. In addition, at least one of the heating members 520 may generate heat. For example, the heating member 520 may include a micro heater.

The connection terminal 530 may electrically connect the heating member 520 with the control module 340. For this, the connection terminal 530 may be placed on the supporting member 510. Also, the connection terminal 530 may transmit a control instruction from the control module 340 to the heating member 520. According to one example embodiment, a plurality of connection terminals 530 may connect a plurality of heating members 520 with the control module 340, respectively. Through this, at least one among the connection terminal 530 may transmit the control instruction to at least one among the heating members 520.

The expanding member 540 may cover the heating member 520. For this, the expanding member 540 may cover the supporting member 510 with placing the heating member 520 therebetween. In addition, the expanding member 540 may be expanded based on heat generated from the heating member 520. Here, the expanding member 540 may be expanded by opposing to the drug cartridge 550 as shown in FIG. 5D. For example, the expanding member 540 may be configured with polymer material.

The drug cartridge 550 may be layered on the heating member 520 and the expanding member 540. In other words, the drug cartridge 550 may be placed on the heating member 520 with placing the expanding member 540 therebetween. The drug cartridge 550 may be storing a fluid form drug f. At this time, the drug cartridge 550 may be storing the drug f in interior space. According to one example embodiment, the drug cartridge 550 may be storing the drug f in one interior space. According to another example embodiment, the interior space may be divided into a plurality of areas, the areas spaced apart from each other, and the drug cartridge 550 may be storing the drug f in each area. Here, the areas may be placed on the plurality of heating members 520, respectively. For example, the same drug f may be stored in at least two among the areas. As another example, the drug f stored in any one among the areas and the drug f stored in another one among the areas may be different. Also, the drug cartridge 550 may supply the drug f to the probe module 330. At this time, as the expanding member 540 is expanded, pressure may be applied to the drug cartridge 550 from the expanding member 540. The drug cartridge 550 may be deformed based on the pressure applied from the expanding member 540. Here, the drug cartridge 550 may be deformed between the supporting member 510 and the probe module 330 as shown in FIG. 5D. Through this, the interior space of the drug cartridge 550 is reduced, and the drug f may be output to the probe module 330 from the interior space of the drug cartridge 550.

The drug cartridge 550 may include at least one projection 551. The projection 551 may be projected toward the probe module 330 in order to be connected to the probe module 330. At this time, the projection 551 may be placed corresponding to the heating member 520. According to one example embodiment, the drug cartridge 550 may include a plurality of projections 551, and the plurality of projections 551 may be placed corresponding to the plurality of heating members 520, respectively. A through-hole 553 may be formed in each of the projections 551. The through-hole 553 may be connected to the probe module 330 from the interior space of the drug cartridge 550. Here, the through-hole 553 may be opened toward the probe module 330. According to one example embodiment, even when the drug cartridge 550 includes a plurality of projections 551, the through-holes 553 of the projections 551 may be respectively extended from the interior space of the drug cartridge 550. According to another example embodiment, in the case that the interior space of the drug cartridge 550 is divided into a plurality of areas, the through-holes 553 of the projections 551 may be respectively extended from the areas of the drug cartridge 550. If pressure is applied from the expanding member 540, the drug cartridge 550 may be deformed between the supporting member 510 and the probe module 330. Through this, as shown in FIG. 5D, as the interior space of the drug cartridge 550 is reduced, the drug f may be output to the through-hole 553 from the interior space of the drug cartridge 550.

The probe module 330 may include at least one among a connection member 560, a probe member 570, at least one light-emitting element 580 or at least one connection terminal 590, as shown in FIG. 5C.

The connection member 560 may be provided for attaching and detaching of the cartridge module 320 in the probe module 330. In other words, the connection member 560 may be substantially connected with the cartridge module 320. At this time, the connection member 560 may be connected with the drug cartridge 550. For this, at least one connection-hole 561 for accepting the projection 551 of the drug cartridge 550 may be formed in the connection member 560. The connection-hole 561 may penetrate the connection member 560. In other words, as at least part of the projection 551 is inserted to the connection-hole 561, the drug cartridge 550 may be connected to the connection member 560. Here, the through-hole 553 of the projection 551 may be exposed inside of the connection-hole 561.

The probe member 570 may be layered on the connection member 560. Here, the probe member 570 may cover the connection-hole 561 of the connection member 560. Also, the probe member 570 may include a neural probe 571 led out to outside from the probe member 570. The neural probe 571 may be configured with flexible material. In addition, the neural probe 571 may be implemented to input stimulation to a predetermined location of tissue of an animal through one end. For this, one end of the neural probe 571 may be opened. Through this, the probe member 570 may input chemical stimulation to the determined location of the tissue by using the drug f supplied from the cartridge module 320. For example, the width of the neural probe 571 may be about 80 μm.

In the neural probe 571, at least one fluid tube 573 may be provided. The fluid tube 573 may be extended along the neural probe 571. At this time, the fluid tube 573 may be connected from the interior space of the drug cartridge 550. For this, the fluid tube 573 may engage with the through-hole 553 of the drug cartridge 550 inside of the connection-hole 561 of the connection member 560. In other words, the through-hole 553 of the drug cartridge 550 may be connected to the fluid tube 573 from the interior space of the drug cartridge 550. Also, the fluid tube 573 may be opened at one end of the neural probe 571 as shown in FIG. 5E. Through this, if the drug f is input through the through-hole 553 from the drug cartridge 550, the drug f flows along the fluid tube 573, and chemical stimulation by the drug f may be generated at one end of the neural probe 571.

The light-emitting element 580 may be mounted at one end of the neural probe 571. According to one example embodiment, the probe module 330 may include a plurality of light-emitting elements 580 as shown in FIG. 5F, and the light-emitting elements 580 may be respectively mounted on different surfaces of the neural probe 571. Also, the light-emitting element 580 may generate light based on a control instruction of the control module 340. At this time, the light-emitting element 580 may generate light from an electrical signal. Through this, optical stimulation by light may be generated at one end of the neural probe 571. For example, the light-emitting element 580 may include μ-ILED (micro inorganic light emitting diode). According to one example embodiment, the plurality of light-emitting elements 580 may respectively generate light in different frequency bands.

It is not shown, but the light-emitting element 580 may be replaced as a power generating element. In addition, the power generating element may generate an electrical signal based on the control instruction of the control module 340. At this time, the power generating element may output the electrical signal. Through this, electrical stimulation may be generated at one end of the neural probe 571.

The connection terminal 590 may electrically connect the light-emitting element 580 with the control module 340. For this, the connection terminal 590 may be mounted on the probe member 570. At this time, the connection terminal 590 may be extended along the neural probe 571. Also, the connection terminal 590 may deliver the control instruction to the light-emitting element 580 from the control module 340. According to one example embodiment, a plurality of connection terminals 590 may connect the plurality of light-emitting elements 580 with the control module 340, respectively. Through this, at least one among the connection terminals 590 may deliver the control instruction to at least one among the light-emitting elements 580.

In some example embodiments, the probe member 570 may further include at least one sensor (not shown). At this time, the sensor may be mounted on the neural probe 571. Here, the sensor may be mounted at one end of the neural probe 571. Also, the sensor may sense a change for stimulation in tissue of an animal. For example, the sensor may sense a change for stimulation based at least one among the chemical method, the optical method or the electrical method. In this case, a part of the connection terminal 590 may electrically connect the sensor with the control module 340. Through this, the control module 340 may acquire a change for stimulation in the animal's brain as sensing data, from the sensor. Also, the control module 340 may transmit the sensing data to the wireless control device 120.

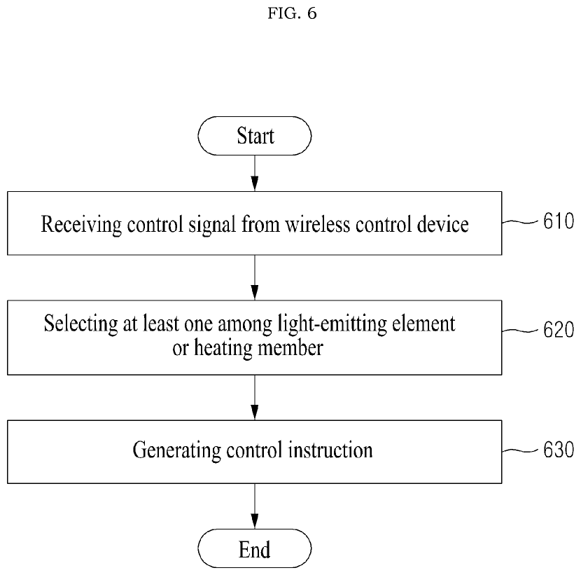

FIG. 6 is a flow chart illustrating an operating method of the neural implant device 200 according to various embodiments.

Referring to FIG. 6, the neural implant device 200 of the electronic device 110 according to various embodiments may receive a control signal from an external device, i.e., the wireless control device 120, in operation 610. At this time, the neural implant device 200 may be connected to the housings 210, 220, 230, and may be fixed on an animal's body through the housings 210, 220, 230. In addition, the neural implant device 200 may be implanted in tissue of an animal. In other words, the neural probe 571 may be led out to a predetermined location of the tissue from the neural implant device 200. Meanwhile, the neural implant device 200 may wirelessly connect with the wireless control device 120. Here, the processor 345 may wirelessly connect with the wireless control device 120 through the communication module 341. For example, the processor 345 may wirelessly connect with the wireless control device 120 based on at least one among BLE (Bluetooth low Energy), Bluetooth, Wi-Fi or IrDA (Infrared Data Association). Through this, the processor 345 may receive a control signal from the wireless control device 120 through the communication module 341.

The neural implant device 200 may select at least one among the light-emitting element 580 or the heating member 520 in operation 620. The processor 345 may select at least one among the light-emitting element 580 or the heating member 520 based on the control signal. According to one example embodiment, in case that the cartridge module 320 includes a plurality of heating members 520, the processor 345 may select at least one among the heating members 520 based on the control signal. According to another example embodiment, in case that the probe module 330 includes a plurality of light-emitting elements 580, the processor 325 may select at least one among the light-emitting elements 580 based on the control signal. According to another example embodiment, in case that the probe module 330 includes a plurality of power generating elements (not shown), the processor 325 may select at least one among the power generating elements based on the control signal.

The neural implant device 200 may generate a control instruction in operation 630. The processor 345 may generate a control instruction for operating at least one of the selected light-emitting element 580 or the selected heating members 520. At this time, the processor 345 may generate a control instruction by using electric energy of the power module 310. According to one example embodiment, the processor 345 may deliver the control instruction to the selected heating member 520. Due to this, the selected heating member 520 generates heat, and through the connection-hole 561 of the projection 551 corresponding to the selected heating member 520, the fluid form of drug f may be supplied to the fluid tube 573 of the neural probe 571 from the drug cartridge 550. Through this, the drug f may be flow along the fluid tube 573, and the chemical stimulation by the drug f may be generated at one end of the neural probe 571. According to another example embodiment, the processor 345 may deliver the control instruction to the selected light-emitting element 580. Due to this, the selected light-emitting element 580 may generate light, and through this, the optical stimulation by light may be generated at one end of the neural probe 571. According to another example embodiment, the processor 345 may deliver the control instruction to the selected power generating element (not shown). Due to this, the selected power generating element may generate light. Through this, the electrical stimulation by an electrical signal may be generated at one end of the neural probe 571.

In some example embodiments, the processor 345 may acquire sensing data from the probe module 330. At this time, a sensor mounted on the neural probe 571 may sense a change for stimulation in tissue of an animal, and deliver it to the processor 345. Through this, the processor 345 may acquire sensing data from the sensor mounted on the neural probe 571. Also, the processor 345 may transmit the sensing data to the wireless control device 120.

In some example embodiments, the processor 345 may store records related to at least one among the control signal, the control instruction or the sensing data, in the memory 343. At this time, the processor 345 may record at least one among the selected light-emitting element 580 or the selected heating member 520 and operating time thereof. For example, the processor 345 may store at least one among the control signal or the control instruction by corresponding to at least one among the selected light-emitting element 580 and the selected heating member 520. As another example, the processor 345 may further store the sensing data by corresponding to at least one among the selected light-emitting element 580 or the selected heating member 520.

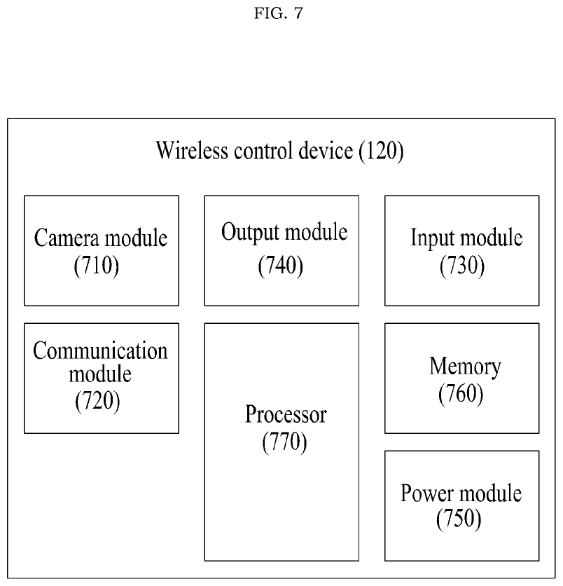

FIG. 7 is a block diagram illustrating the wireless control device 120 according to various embodiments.

Referring to FIG. 7, an electronic device according to various embodiments, i.e., the wireless control device 120 may include at least one among a camera module 710, a communication module 720, an input module 730, an output module 740, a power module 750, a memory 760 or a processor 770. In some example embodiments, at least one among the components of the wireless control device 120 may be omitted, and at least one another component may be added. In some example embodiments, at least two among the components of the wireless control device 120 may be implemented as one integrated circuit.

The camera module 710 may photograph surrounding images. For example, the camera module 710 may be installed in a predetermined location, and photograph images. Also, the camera module 710 may generate image data for the images. For example, the camera module 710 may include at least one among a lens, at least one image sensor, an image signal processor or a flash.

The communication module 720 may support communication between the wireless control device 120 and an external device. For example, the external device may include at least one among the neural implant device 200, another electronic device, a base station, and a satellite. At this time, the communication module 720 may include at least one among a wireless communication module or a wire communication module. According to one example embodiment, the wireless communication module may support at least one among a long distance communication method or a near field communication method. The near field communication method may include at least one among BLE (Bluetooth low Energy), Bluetooth, Wi-Fi or IrDA (Infrared Data Association). The wireless communication method may communicate with the long distance communication method through a network, and the network may include at least one among e.g., a cellular network, the Internet or a computer network such as LAN (Local Area Network) or WAN (Wide Area Network). According to another example embodiment, the wireless communication module may support communication with GNSS (Global Navigation Satellite system). For example, GNSS may include GPS (Global Positioning System).

The input module 730 may receive an instruction or data to be used to at least one among the components of the wireless control device 120 from outside of the wireless control device 120. For example, the input module 730 may include at least one among a microphone, a mouse or a keyboard. In some example embodiments, the input module may include at least one among a touch circuitry set to sense touch or a sensor circuitry set to measure force generated by touch.

The output module 740 may provide information to the outside of the wireless control device 120. At this time, the output module 740 may include at least one among a display module or an audio module. The display module may visually output information. For example, the display module may include at least one among a display, a hologram device, or a projector. In some example embodiments, the display module may be implemented as a touch screen by being assembled with at least one among the touch circuitry or the sensor circuitry. The audio module may output information into sound. For example, the audio module may include at least one among a speaker or a receiver.

The power module 750 may manage electric energy supplied to at least one component of the wireless control device 120. According to one example embodiment, the power module 750 may include at least one among a battery module for storing electric energy or a wireless charge module for charging the battery module or a wireless power management module which enables direct use of energy wirelessly transmitted without the need for the battery module. For example, the battery module may include at least one among a primary cell which may not be recharged, a second cell which may be recharged or a fuel cell.

The memory 760 may store various data used by at least one component of the wireless control device 120. For example, the memory 760 may include at least one among volatile memory or nonvolatile memory. Data may include at least one program and input data or output data related thereto. The program may be stored as software including at least one instruction in the memory 760, and may include at least one among an operating system, a middleware or an application. According to one example embodiment, the application may be for controlling the neural implant device 200.

The processor 770 may control at least one component of the wireless control device 120 by executing the program of the memory 760. Through this, the processor 345 may perform data process or operation. The processor 770 may determine stimulation for generating on tissue of an animal, and generate a control signal therefor. According to one example embodiment, the processor 770 may monitor an animal in which the neural implant device 200 is implanted, and based on the result, may determine a control signal. At this time, the processor 770 may analyze the animal's movement. For example, the processor 770 may monitor the animal's movement through the camera module 710. As another example, the processor 770 may monitor the animal's movement through the communication module 720. For this, another electronic device such as a sensor device may be attached to the animal's body and sense the body's change, and the processor 770 may monitor the animal's movement by communicating with the another electronic device through the communication module 720. Also, the processor 770 may determine stimulation based on the animal's movement, and may generate a control signal therefor. According to another example embodiment, the processor 770 may determine a control signal based on a UI (User Interface). At this time, the processor 770 may determine stimulation selected by a user through the UI, and may generate a control signal therefor. Through this, the processor 770 may transmit the control signal to the neural implant device 200 through the communication module 720.

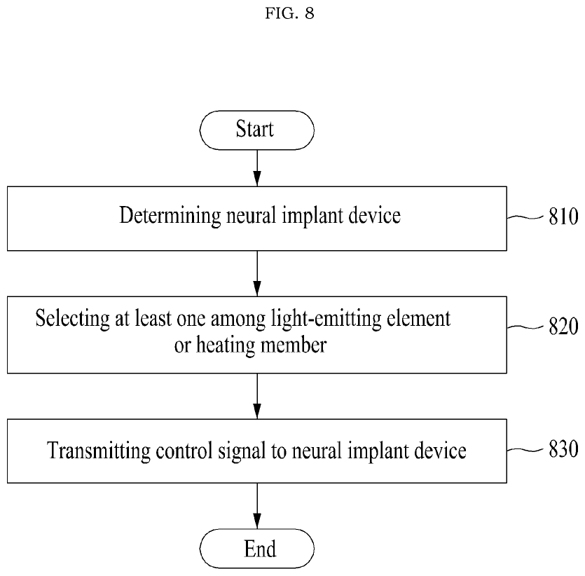

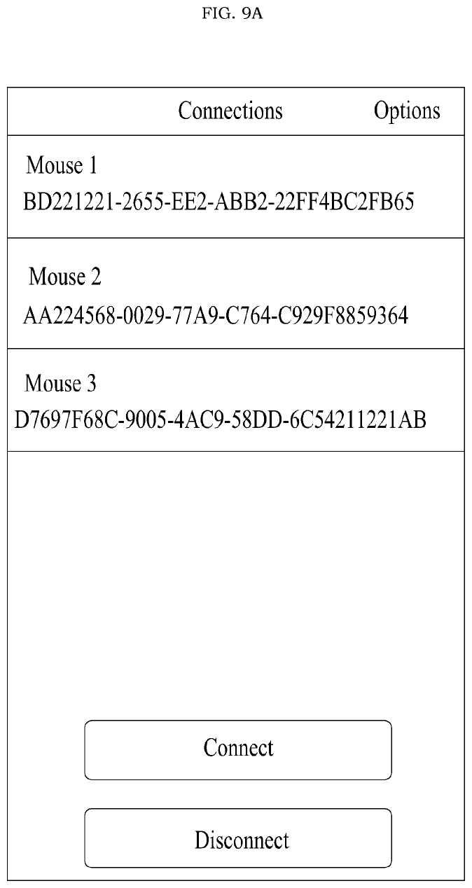

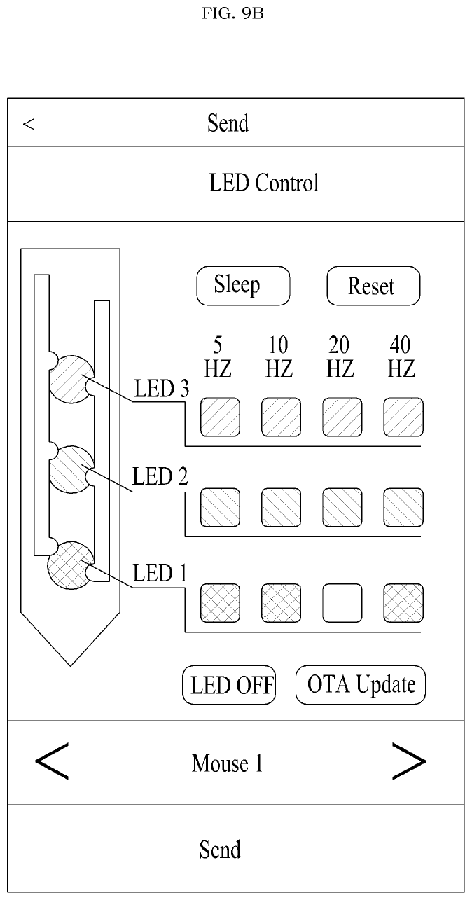

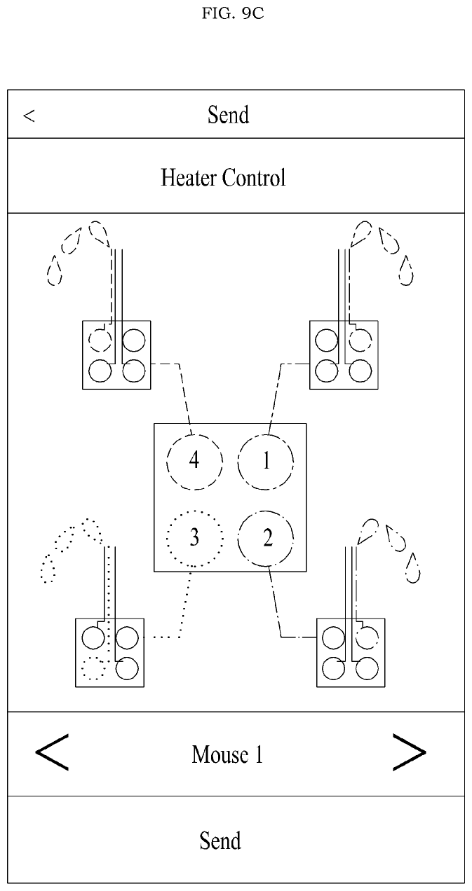

FIG. 8 is a flow chart illustrating an operating method of the wireless control device 120 according to various embodiments, and FIGS. 9A, 9B, and 9C are example drawings illustrating a user interface of the wireless control device 120 according to various embodiments.

Referring to FIG. 8, an electronic device according to various embodiments, i.e., the wireless control device 120 may determine the neural implant device 200 in operation 810. At this time, in the wireless control device 120, at least one controllable neural implant device 200 may be registered in advance. Here, in the memory 760, identification information of the neural implant device 200 may be stored. The processor 770 may determine the neural implant device 200. According to an example embodiment, the processor 770 may determine an animal that needs stimulation and the neural implant device 200 implanted therein, based on the monitoring result for the animal. According to another example embodiment, the processor 770 may display a screen for selecting the neural implant device 200 through a user interface as shown in FIG. 9, and may select the neural implant device 200 based on input of a user. Also, the processor 770 may wirelessly connect with the determined neural implant device 200 through the communication module 720. Here, the processor 770 may connect with the neural implant device 200 determined based on, e.g., BLE.

The wireless control device 120 may select at least one among the light-emitting element 580, the power generating element (not shown) or the heating member 520 in operation 820. The processor 770 may select at least one among the light-emitting element 580, the power generating element or the heating member 520 of the determined implant device 200. According to one example embodiment, the processor 770 may select at least one among the light-emitting element 580, the power generating element or the heating member 520 based on the monitoring result for the animal. According to another example embodiment, the processor 770 may display a screen for selecting at least one among the light-emitting element 580, the power generating element or the heating member 520 through a user interface as shown in FIG. 9B or 9C, and may select at least one among the light-emitting element 580, the power generating element or the heating member 520 based on input of a user. For example, the processor 770 may display a screen for selecting at least one among a plurality of light-emitting elements 580 as shown in FIG. 9B. Through this, a user may select at least one among the light-emitting elements 580. Here, the user may further select operating frequency, e.g., the number of light-emitting per second, of the selected light-emitting element 580. As another example, the processor 770 may display a screen for selecting at least one among a plurality of heating members 520 as shown in FIG. 9C. Through this, the user may select at least one among the heating members 520. Here, it is not shown, but the user may further select the heating time of the heating member 520.

The wireless control device 120 may transmit the control signal to the neural implant device 200 determined in operation 830. The processor 770 may generate the control signal based on at least one among the determined neural implant device 200 and the selected heating member 520 or the selected light-emitting element 580. In addition, the processor 770 may transmit the control signal to the neural implant device 200 through the communication module 720. Here, the processor 770 may transmit the control signal to the determined neural implant device 200 based on e.g. BLE.

According to various embodiments, the electronic device 110 is attached to the animal's body, but the neural probe 571 may be substantially implanted in nervous tissue of the animal. As the neural probe 571 is led out to a predetermined location of the nervous tissue of the animal, the neural implant device 200 of the electronic device 110 may elaborately deliver stimulation to the corresponding location. At this time, the neural probe 571 may deliver at least one among chemical stimulation by a fluid form of drug, optical stimulation by light or electrical stimulation by an electrical signal. Here, selective chemical stimulation based on various drugs may be delivered, and selective optical stimulation based on light of various frequency ranges may be delivered. Also, since the neural probe 571 is formed with flexible material and with a width of about 80 μm, even if the neural probe 571 is implanted in the tissue of the animal, nervous tissue damage and inflammatory response of the animal may be minimized. In addition, as the cartridge module 320 in which drug is stored is implemented to be detachable to the neural implant device 200, the cartridge 320 may be replaceable. Due to this, the neural implant device 200 may be used for a long time while implanted in the animal's tissue. Also, since the neural implant device 200 may be wirelessly controlled by using an external device such as a smartphone, i.e., the wireless control device 120, the use efficiency of the neural implant device 200 may be increased.

The electronic device 110 according to various embodiments, which is for delivering stimulation to tissue by being implanted in the tissue of an animal, may be formed with flexible material, include the neural probe 571 led out to a predetermined location of the tissue, and include the probe module 330 configured to input the stimulation to the location through the neural probe 571 and the control module 340 connected to the probe module 330 and configured to generate a control instruction for generating the stimulation.

According to various embodiments, the stimulation may include chemical stimulation by a fluid form of drug, and the neural probe 571 may include at least one fluid tube 573 for inputting the chemical stimulation to the location by being formed to flow the drug and opened at one end of the neural probe 571.

According to various embodiments, the stimulation may include at least one among optical stimulation by light or electrical stimulation by an electrical signal, and the probe module 330 may be mounted on one end of the neural probe 571, and based on the control instruction, may further include at least one element (e.g., the light-emitting element 580 and the power generating element (not shown)) for generating at least one among the optical stimulation or the electrical stimulation to the location, and at least one connection terminal 590 for electrically connecting the element (e.g., the light-emitting element 580 and the power generating element (not shown)) and the control module 340 by extending along the neural probe 571.

According to various embodiments, the electronic device 110 may further include the cartridge module 320 connected to the control module 340, storing the drug, and configured to supply the drug to the fluid tube 573 based on the control instruction.

According to various embodiments, the cartridge module 320 may be implemented to be detachable to the probe module 330.

According to various embodiments, the cartridge module 320 may include the drug cartridge 550 storing the drug in interior space, projecting toward to the probe module 330 in order to be connected to the probe module 330, and including at least one projection 551 in which the through-hole 553 connected to the fluid tube 573 from the interior space is formed.

According to various embodiments, the cartridge module 320 may be implemented to output the drug to the fluid tube 573 from the interior space through the through-hole 553 based on at least one among a heat-based fluidic pump method, a magnetically-actuated fluidic pump method, a shape memory alloy or shape memory polymer substrate fluidic pump method, or an electrochemistry fluidic pump method.

In some example embodiments, based on the control instruction, the cartridge module 320 may further include the at least one heating member 520 for generating heat and the expanding member 540 for applying pressure to the drug cartridge 550 by expanding based on the heat, and the drug cartridge 550 may output the drug to the fluid tube 573 from the interior space through the through-hole 553 based on the pressure.

According to various embodiments, the probe module 330 may include the connection member 560 in which the at least one connection-hole 561 for accepting the projection 551 is formed.

According to various embodiments, the neural probe 571 may be led out from the connection member 560, and the fluid tube 573 may engage with the through-hole 553 at inside of the connection-hole 561.

According to various embodiments, the control module 340 may be configured to wirelessly receive a control signal from the external device 120, and based on the control signal, to generate the control instruction.

According to various embodiments, the control module 340 may be configured to wirelessly communicate with the external device 120 based on BLE.

According to various embodiments, the external device 120 may be configured to provide a user interface for controlling the electronic device, and based on the user interface, to generate the control signal.

According to various embodiments, the electronic device 110 may further include the battery module 310 configured to generate electric energy.

According to various embodiments, the control module 340 may be configured to be connected to the battery module 310, and to generate the control instruction by using the electric energy.

According to various embodiments, the probe module 330 and the control module 340 may be combined with the housings 210, 220, 230 fixed on the animal's body.

According to various embodiments, the neural probe 571 may be led out to the location from the probe module 330.

An operating method of the electronic device 110 according to various embodiments, which is for delivering stimulation to nervous tissue by being implanted in the nervous tissue of an animal, may include generating a control instruction based on a control signal wirelessly received from the external device 120, and inputting the stimulation to a location, through the neural probe 571 formed with flexible material and led out to the predetermined location of the tissue based on the control instruction.

According to various embodiments, the stimulation may include chemical stimulation by a fluid form of drug, and the inputting of the stimulation may comprise inputting the chemical stimulation to the location by flowing the drug through the at least one fluid tube 573 formed to be extended along the neural probe 571 and opened at one end of the neural probe 571.

According to various embodiments, the stimulation may include at least one among optical stimulation by light or electrical stimulation by an electrical signal, and the inputting of the stimulation may include at least one among the optical stimulation or the electrical stimulation to the location through at least one element (e.g., the light-emitting element 580 and the power generating element (not shown)) mounted on one end of the neural probe 571.

According to various embodiments, the operating method of the electronic device 110 may further include wirelessly connecting with the external device 120 based on at least one among BLE, Bluetooth, Wi-Fi, or infrared communication.

According to various embodiments, the electronic device 110 may be configured to provide a user interface for controlling the electronic device 110, and generate the control signal based on the user interface.

According to various embodiments, the electronic device 110 may include the battery module 310 configured to generate electric energy.

According to various embodiments, the generating of the control instruction may include an operation for generating the control instruction by using the electric energy.

According to various embodiments, the electronic device 110 may be connected to the housings 210, 220, 230 fixed on the animal's body, and at least one among components in the electronic device 110 may be implemented to be detachable to at least another one among the components.

It should be understood that various embodiments of the disclosure and terms used in the embodiments do not intend to limit technical features disclosed in the disclosure to the particular embodiment disclosed herein; rather, the disclosure should be construed to cover various modifications, equivalents, or alternatives of embodiments of the disclosure. With regard to description of drawings, similar or related components may be assigned with similar reference numerals. As used herein, singular forms of noun corresponding to an item may include one or more items unless the context clearly indicates otherwise. In the disclosure disclosed herein, each of the expressions “A or B”, “at least one of A and B”, “at least one of A or B”, “A, B, or C”, “one or more of A, B, and C”, or “one or more of A, B, or C”, and the like used herein may include any and all combinations of one or more of the associated listed items. The expressions, such as “a first”, “a second”, “the first”, or “the second”, may be used merely for the purpose of distinguishing a component from the other components, but do not limit the corresponding components in the importance or the order. It is to be understood that if an element (e.g., a first element) is referred to as “coupled to (functionally or communicatively)” or “connected to” another element (e.g., a second element), it means that the element may be coupled with the other element directly, or via the other element (e.g., a third element).

The term “module” used in the disclosure may include a unit implemented in hardware, software, or firmware and may be interchangeably used with the terms logic, logical block, part, or circuit. The module may be a minimum unit of an integrated part or may be a part thereof. The module may be a minimum unit for performing one or more functions or a part thereof. For example, the module may include an application-specific integrated circuit (ASIC).

According to various embodiments, each component (e.g., the module or the program) of the above-described components may include one or plural entities. According to various embodiments, at least one or more components of the above components or operations may be omitted, or one or more components or operations may be added. Alternatively or additionally, some components (e.g., the module or the program) may be integrated in one component. In this case, the integrated component may perform the same or similar functions performed by each corresponding components prior to the integration. According to various embodiments, operations performed by a module, a programming, or other components may be executed sequentially, in parallel, repeatedly, or in a heuristic method, or at least some operations may be executed in different sequences, omitted, or other operations may be added.

The embodiments of the invention in which an exclusive property or privilege is claimed are defined as follows:

1. An electronic device implanted in neural tissue of an animal and for delivering stimulation to the tissue, comprising:
a probe module including a neural probe formed with flexible material and led out to a predetermined location of the tissue, and configured to input the stimulation to the location through the neural probe; and
a control module configured to be connected to the probe module and generate a control instruction for occurring the stimulation.

2. The device of claim 1,
wherein the stimulation comprises chemical stimulation by a fluid form of drug, and
wherein the neural probe comprises at least one fluid tube for inputting the chemical stimulation to the location by being formed to flow the drug and opened at one end of the neural probe.

3. The device of claim 1,
wherein the stimulation comprises at least one among optical stimulation by light or electrical stimulation by an electrical signal, and
wherein the probe module further comprises:
at least one element for generating at least one of the optical stimulation or the electrical stimulation on the location based on the control instruction by being mounted on one end of the neural probe; and
at least one connecting terminal prolonged along the neural probe and for electrically connecting the element and the control module.

4. The device of claim 2 further comprising:
a cartridge module configured to be connected to the control module, store the drug, and supply the drug to the fluid tube based on the control instruction.

5. The device of claim 4, wherein the cartridge module is implemented to be detachable to the probe module.

6. The device of claim 4, wherein the cartridge module comprises:
at least one projection storing the drug in interior space, projecting toward the probe module in order to be connected to the probe module, and forming a through-hole connected to the flow tube from the interior space.

7. The device of claim 6, wherein the cartridge module is implemented to output the drug to the fluid tube from the interior space through the through-hole based on any one among a heat-based fluidic pump method, a magnetically-actuated fluidic pump method, a shape memory alloy or shape memory polymer substrate fluidic pump method, or an electrochemistry fluidic pump method.

8. The device of claim 6, wherein the probe module comprises:
connecting members formed at least one connecting hole for accepting the projection, and
wherein the neural probe is led out from the connecting members and the fluid tube engages with the through-hole at the inside of the connecting hole.

9. The device of claim 1, wherein the control module is configured to wirelessly receive a control signal from an external device, and generate the control instruction based on the control signal.

10. The device of claim 9, wherein the control module is configured to wirelessly communicate with the external device based on at least one of Bluetooth Low Energy (BLE), Bluetooth, Wi-Fi, or infrared communication.

11. The device of claim 9, wherein the external device is configured to provide a user interface for controlling the electronic device, and generate the control signal based on the user interface.

12. The device of claim 9 further comprising:
a battery module,
wherein the control module is configured to be connected with the battery module, and generate the control instruction by using the electric energy.

13. The device of claim 1,
wherein the probe module and the control module are connected with a housing fixed on the animal's body, and
wherein the neural probe is led out to the location from the probe module.

14. An operating method of an electronic device implanted in tissue of an animal and for delivering stimulation to the tissue comprising:
generating a control instruction based on a control signal wirelessly received from an external device; and
inputting the stimulation to a location through a neural probe which is formed with flexible material and led out to the predetermined location of the tissue, based on the control instruction.

15. The method of claim 14,
wherein one or more wireless signals are generated from a user-controlled or closed-loop (automatic or pre-programmed) transmitter, that can be sent to one or more selectively-chosen receivers within a large group of wireless receivers with high accuracy and reliability.

16. The method of claim 14,
wherein the method is performed by multiple closed loop systems where a certain tethered or untethered trigger in a transmitter can broadcast multiple wireless signals simultaneously, each with a unique key, and all receivers in the vicinity after receiving all signals will be able to decode only those signals matching with the security keys pre-programmed in their firmware, and then each receiver will process specific one or more functionalities based on the validated signal it decoded.

17. The method of claim 14,
wherein the stimulation comprises chemical stimulation by a fluid form of drug, and
wherein the inputting of the stimulation comprises:
inputting the chemical stimulation to the location by flowing the drug through at least one fluid tube formed to be prolonged along the neural probe and opened at one end of the neural probe.

18. The method of claim 14,
wherein the stimulation comprises at least one among optical stimulation by light or electrical stimulation by an electrical signal, and
wherein the inputting of the stimulation comprises:
inputting at least one among the optical stimulation or the electrical stimulation to the location through at least one element mounted on one end of the neural probe.

19. The method of claim 14,
wherein the external device is configured to provide a user interface for controlling the electronic device, and generate the control signal based on the user interface,
wherein the electronic device comprises a battery module configured to generate electric energy, and
wherein the generating of the control instruction comprises:
generating the control instruction by using the electric energy.

20. The method of claim 14, wherein the electronic device is connected to a housing fixed on the animal's body, and
wherein at least one of components in the electronic device is implemented to be detachable for at least another one of the components.
